(19) United States
(12) Patent Application Publication
(10) Pub. No.: US 2005/0059970 A1
(43) Pub. Date: Mar. 17, 2005

(54) Bone fixation systems

(76) Inventor: Eric Kolb, Quincy, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/664,199

(22) Filed: Sep. 17, 2003

(51) Int. Cl. A61B 17/58

(52) U.S. Cl. 606/69

(57) ABSTRACT

A bone fixation system may include a bone anchor having a proximal head and a distal portion configured to engage bone and a plate having at least one hole for receiving the bone anchor. At least one hole of the plate includes a plurality of concentric annular bores formed in the plate and at least one of the plurality of concentric annular bores is sized and shaped to engage the proximal head of the bone anchor to facilitate coupling of the bone anchor to the plate.

### BACKGROUND

Advancing age, as well as injury, can lead to changes in the bones, discs, joints, and ligaments of the spine, producing pain from nerve compression. Under certain circumstances, alleviation of pain can be provided by performing spinal fusion. Spinal fusion is a procedure that generally involves the removal of the disc between two or more adjacent vertebrae and the subsequent joining of the vertebrae with a bone fixation device to facilitate growth of new osseous tissue between the vertebrae. The new osseous tissue fuses the joined vertebrae such that the vertebrae are no longer able to move relative to each other. Bone fixation devices can stabilize and align the injured bone segments to ensure the proper growth of the new osseous tissue between the damaged segments. Bone fixation devices are also useful for promoting proper healing of injured or damaged vertebral bone segments caused by trauma, tumor growth, or degenerative disc disease.

One such bone fixation device is a bone fixation plate that is used to stabilize, align, and, in some cases, immobilize adjacent skeletal parts such as bones. Typically, the fixation plate is a rigid metal or polymeric plate positioned to span bones or bone segments that require stabilization, alignment, and/or immobilization with respect to one another. The plate may be fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed or during spinal fusion.

Such plates have been used to stabilize, align, and/or immobilize a variety of bones, including vertebral bodies of the spine. For example, a bone plate may include a plurality of holes for bone anchor placement. The bone plate may be placed against the damaged vertebral bodies and bone screws or other bone anchors can be used to secure the bone plate to the vertebral bodies. In the case of spinal fusion, for example, a prosthetic implant or bone graft may be positioned between the adjacent vertebrae to promote growth of osseous tissue and fusion of the vertebrae.

It is important for the proper functioning of the bone fixation plate that the plate be securely affixed by one or more bone anchors to bone. The secure affixation of the bone fixation plate to bone depends primarily on the achievement of positive locking between the head of the bone anchor and the anchor holes of the plate. Such locking is problematic for smaller size bone fixation plates, particularly plates designed for use in the cervical region of the spine.

### SUMMARY

Disclosed herein are bone fixation systems that facilitate the stabilization, alignment and/or immobilization bone, in particular, one or more vertebral bodies of the spine. The disclosed bone fixation systems provide a locking system that facilitates positive locking of one or more bone anchors to the anchor holes provided in a bone fixation plate. The locking system is particular suited for use with smaller sized bone fixation plates, such as cervical plates, although the locking system may be used with plates of any type, size or shape.

In accordance with one exemplary embodiment, a bone fixation system may comprise a bone anchor having a proximal head and a distal portion configured to engage bone and a plate having at least one hole for receiving the bone anchor. In the exemplary embodiment, the at least one hole of the plate includes a plurality of concentric annular bores formed in the plate and at least one of the plurality of concentric annular bores is sized and shaped to engage the proximal head of the bone anchor to facilitate coupling of the bone anchor to the plate.

In accordance with another exemplary embodiment, a bone fixation system may comprise a bone plate having a plurality of plate holes for receiving a bone anchor therein and a plurality of bone anchors for coupling the bone plate to bone. In the exemplary embodiment, at least one of the bone anchors may have a tapered proximal head and a distal portion configured to engage bone. The proximal head of the at least one anchor may taper toward the distal portion of the bone anchor. In addition, at least one of the plate holes may have a generally stepped-shaped inner wall surface provided by a plurality of steps formed in the inner wall of the at least one plate hole. Preferably, a plurality of the steps have a generally annular peak and a plurality of the peaks within a hole are aligned in a generally frusta-conical shape to facilitate gripping engagement of the tapered proximal head of a bone anchor upon advancement of the bone anchor into the plate hole.

### BRIEF DESCRIPTION OF THE DRAWINGS

These and other features and advantages of the bone fixation systems disclosed herein will be more fully understood by reference to the following detailed description in conjunction with the attached drawings in which like reference numerals refer to like elements through the different views. The drawings illustrate principles of the bone fixation systems disclosed herein and, although not to scale, show relative dimensions.

FIG. 1 is a perspective view of an exemplary embodiment of a single level dynamic bone fixation plate;

FIGS. 2A and 2B are perspective views of the bone fixation plate of FIG. 1, illustrating the connection of a plurality of bone anchors to the plate;

FIG. 3A is a partially schematic side elevational view in cross section of an exemplary embodiment of an anchor hole of the bone fixation plate of FIG. 1;

FIG. 3B is a partially schematic side elevational view in cross section of the radially inner surface of the anchor hole of FIG. 3A;

FIG. 4 is a partially schematic side elevational view in cross section of the anchor hole of FIG. 3A, illustrating a bone anchor positioned within the anchor hole of the plate;

FIG. 5 is a partially schematic side elevational view in cross section of an exemplary embodiment of a bushing;

FIG. 6 is a partially schematic side elevational view in cross section of another embodiment of an anchor hole of a bone fixation plate, illustrating the barbed shaped geometry of the anchor hole;

FIG. 7 is a partially schematic side elevational view in cross section of another embodiment of an anchor hole of a bone fixation plate, illustrating the ridged geometry of the anchor hole;

FIG. 8 is a partially schematic side elevational view in cross section of another embodiment of an anchor hole of a bone fixation plate, illustrating a cut-out within the wall of the anchor hole to inhibit bone anchor back-out; and

FIG. 9 is a partially schematic side elevational view in cross section of an exemplary embodiment of a bone anchor including a locking mechanism.

### DETAILED DESCRIPTION OF EXEMPLARY EMBODIMENTS

Certain exemplary embodiments will now be described to provide an overall understanding of the principles of the structure, function, manufacture, and use of the bone fixation systems disclosed herein. One or more examples of these embodiments are illustrated in the accompanying drawings. Those of ordinary skill in the art will understand that the bone fixation systems specifically described herein and illustrated in the accompanying drawings are non-limiting exemplary embodiments and that the scope of the present invention is defined solely be the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

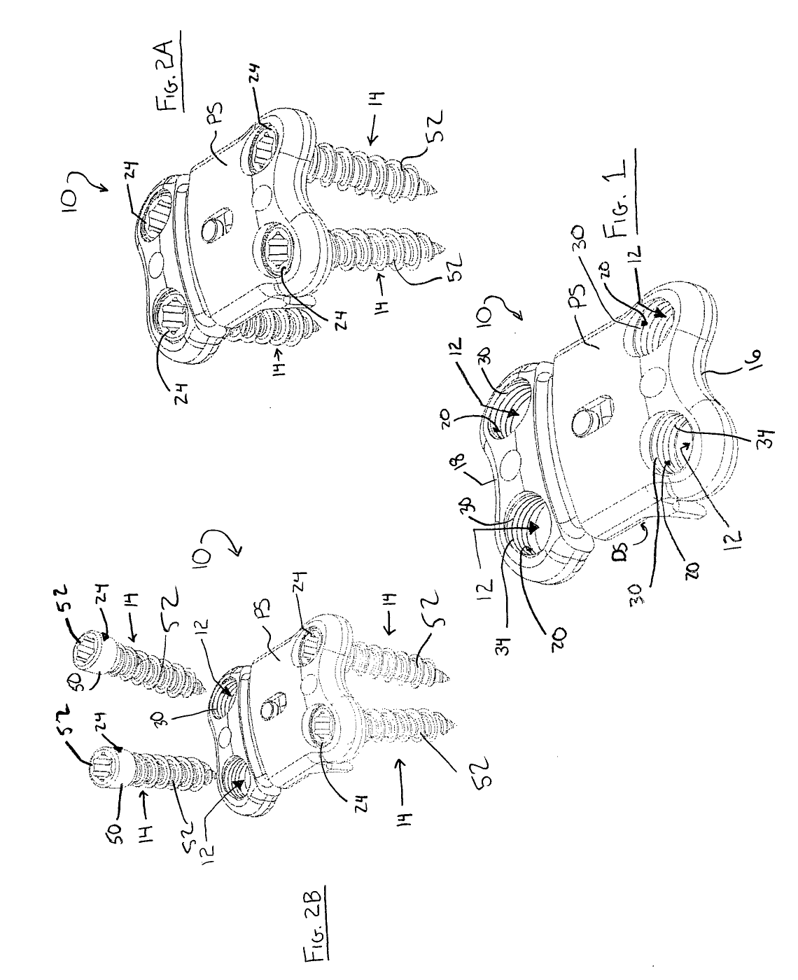

FIGS. 1-2A illustrate an exemplary embodiment of a single level dynamic bone fixation plate 10. The exemplary bone fixation plate 10 is designed to stabilize and align two adjacent bone segments, in particular, two adjacent vertebral bodies. When implanted, the exemplary bone fixation plate 10 may be fixed at opposing ends to the two adjacent vertebral bodies and extend over the disc space between the adjacent vertebral bodies. Although the exemplary bone fixation plate 10 described below is designed primarily for use in spinal applications, such as to stabilize and align adjacent vertebrae to facilitate fusion of the vertebrae, one skilled in the art will appreciate that the structure, features, and principles of the exemplary bone fixation plate 10, as well as the other exemplary embodiments described below, may be applied to any fixation device used to connect two or more sections of bone. Non-limiting examples of applications of the bone fixation plates described herein include long bone fracture fixation/stabilization, small bone stabilization, lumbar spine as well as thoracic stabilization/fusion, cervical spine compression/fixation, and skull fracture/reconstruction plating.

The bone fixation plate 10 has a distal surface (DS) that faces and engages the bone surface upon implantation of the plate and a proximal surface (PS) that faces away from the bone surface and is opposite the distal surface. The term “distal” as used herein with respect to any component or structure will generally refer to a position or orientation that is proximate, relatively, to the bone surface to which bone plate is to be applied. Conversely, the term “proximal” as used herein with respect to any component or structure will generally refer to a position or orientation that is distant, relatively, to the bone surface to which bone plate is to be applied.

The structure and function of the exemplary single level dynamic bone fixation plate is described in detail in commonly owned, co-pending U.S. patent application Ser. No. ______, filed concurrently herewith, entitled Bone Fixation Plates (Attorney Docket No. DEP 5156), which is incorporated herein in by reference. One skilled in the art will appreciate that the locking systems disclosed herein and described in detail below may be incorporated in any type or size bone fixation plate, including both rigid and dynamic plates, as well as any other bone fixation devices.

Referring to FIGS. 1-4, the exemplary bone fixation plate 10 includes one or more anchor holes 12 for receiving a bone anchor, such as a bone screw 14, which is effective to mate the bone fixation plate 10 to bone. The bone fixation plate 10 may include any number of anchor holes 12 to fix the plate 10 to bone. The number of anchor holes may vary depending on, for example, the size of the plate, the type(s) of bone anchor(s) employed, and the location and anatomy of bone being secured. In the illustrated exemplary embodiment, the bone fixation plate 10 includes two anchor holes 12 positioned proximate a first end 16 of the plate and two anchor holes 12 positioned proximate a second end 18 of the plate. In the illustrated embodiment, the anchor holes 12 are symmetrically positioned about the longitudinal axis of the bone fixation plate 10 and proximate to the ends 16, 18 of the plate, although one skilled in the art will appreciate that other locations are possible.

The size and shape of each anchor hole 12 is preferably selected to match the size and shape of the selected bone anchor. For example, the radially inner surface 20 of the anchor hole 12 may define an anchor passage 22 for receiving and securely engaging a portion of the bone anchor 14, such as the proximal head 24 of the exemplary bone screw 14. In certain exemplary embodiments, the anchor passage 22 may be complementary in size and shape to the proximal head 24 of the bone anchor 14 to facilitate locking engagement of the proximal head 24 to the inner surface 20 of the anchor hole 12, as discussed below.

Continuing to refer to FIGS. 1-4, one more of the anchor holes 12 may include a locking mechanism that facilitates positive locking of the bone anchor 14 to the anchor hole 12. The locking mechanism in the illustrated exemplary embodiment comprises a plurality of concentric annular bores 30 formed in inner surface 20 of the anchor hole 12. Preferably, at least one of the plurality of concentric annular bores 30 is sized and shaped to engage the proximal head 24 of the bone anchor 14 to facilitate coupling of the bone anchor 14 to the plate 10, as discussed in detail below.

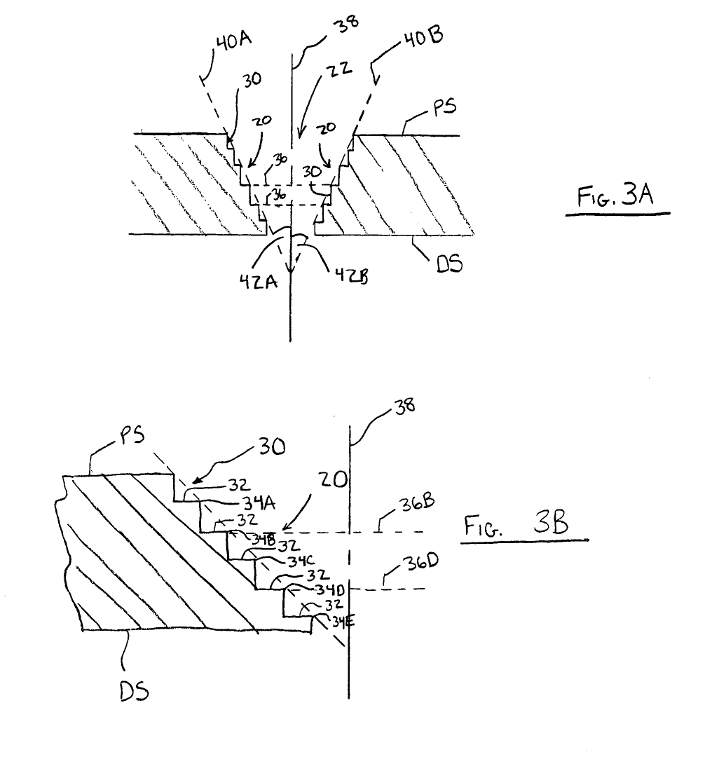

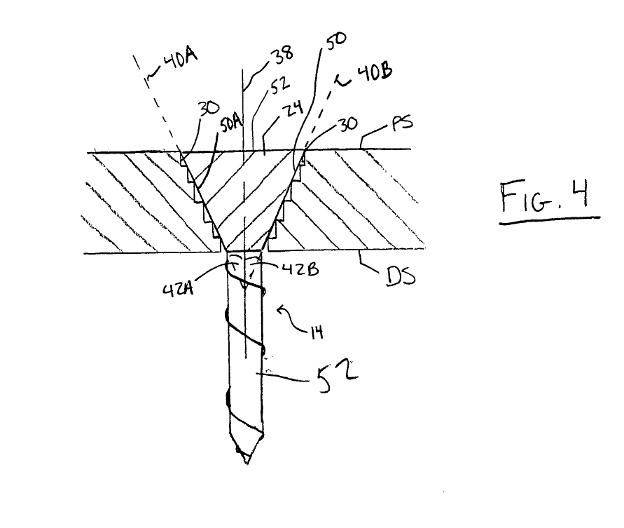

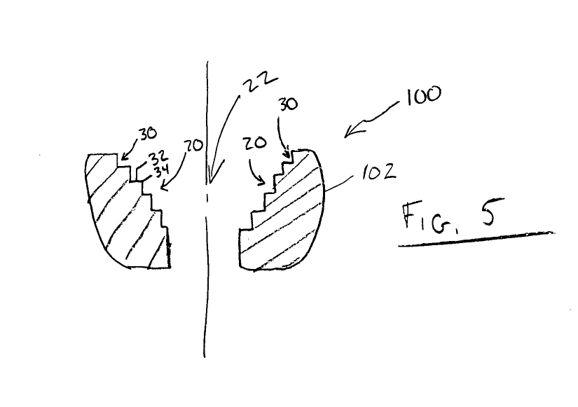

Referring to FIGS. 3A-4, the plurality of concentric annular bores 30 may provide the radial inner surface 20 of exemplary the anchor hole 12 with a generally stepped-shaped configuration. In particular, one or more of the bores 30 may include a radially extending step surface 32 that terminates at a radially inner, annular-shaped peak 34. Each peak 34 can provide an engagement surface for grippingly engaging the proximal head 24 of bone anchor 14. Each of the annular peaks 34 defines a diameter and a peak plane 36. To form the stepped shaped configuration, one or more of the peaks 34 may have a diameter different from the other diameters of the other peaks 24. In the exemplary embodiment illustrated in FIGS. 3A-4, each of the peaks 34A-34E has a diameter that is less than the diameter of the peak 34 that is proximally adjacent to the peak. For example, peak 34B has a diameter that is less than the diameter of peak 34A, peak 34C has a diameter that is less than the diameter of peak 34B, peak 34D has a diameter that is less than the diameter of peak 34C, and peak 34E has a diameter that is less than the diameter of peak 34D.

In the illustrated exemplary embodiment, the peak planes 36 of each peak 34, as well as the step surfaces 32, may be oriented parallel to one another and may intersect, and, preferably, may be perpendicular to, the hole axis 38 of the exemplary anchor hole 12, although, one of ordinary skill in the art will appreciate that other orientations are possible. Other exemplary orientations include embodiments in which one or more of the peaks planes 36 are oriented at angle other than perpendicular to the hole axis 38 and/or embodiments in which one or more of the peak planes 36 are non-parallel with respect to another peak plane.

A plurality of the annular peaks 36 may be aligned to provide a generally frusta-conical shape to the anchor passage 22 of the anchor hole 12, as best illustrated in FIG. 3A. The frusta-conical shaped anchor passage 22 is generally defined, in cross-section, by two intersecting peak axes 40A and 40B. The peak axes 40A, 40B each intersect a plurality of the peaks 34 of the anchor hole 12. In the illustrated embodiment, for example, each peak axis 40A, 40B intersects each of the peaks (34A-34E). The peak axes 40A, 40B are preferably oriented symmetrically about the hole axis 38, although non-symmetrical orientations are possible. For example, each peak axis 40A, 40B may intersect the hole axis 38 at a common angle 42A, 42B. The degree of angulation of the anchor passage 22 may be varied depending upon, for example, the bone anchor employed by adjusting the diameter of one or more peaks 34, and, thus, adjusting the angles 42A, 42B. The peak angle 42, in certain exemplary embodiments, may be 2°-10° with respect to the bore axis 38. Preferably, the peak angle 42 is 3° with respect to the bore axis 38.

The exemplary annular bores 30 may be formed in an anchor hole 12 by machining, casting, and/or molding, or by other conventional processes for manufacturing medical implants.

As discussed above, the anchor passage 22 preferably has a shape that is complementary to the shape of the proximal head 24 of the bone anchor 12. In the case of the exemplary frusta-conical shaped anchor passage 22 described above, the proximal head 24 preferably has a frusta-conically shaped outer surface 50 that tapers distally from a circular shaped proximal end surface 52. The taper angle of the outer surface 50 of the proximal head 24 is preferably generally equal to the peak angles 42A, 42B of the peak axes 40A, 40B, as best illustrated in FIGS. 3A and 4. Thus, as the proximal head 24 of the bone anchor 14 is advanced into the exemplary anchor hole 12, the peaks 34 of the anchor hole 12 grippingly engage the outer surface 50 of the proximal head 24 to facilitate locking engagement of the proximal head 24 to the anchor hole 12.

The outer surface 50 of the proximal head 24 of the exemplary bone anchor 14 is smooth, i.e., the outer surface 50 preferably lacks threads and/or surface texturing. Although, one skilled in art will appreciate that the outer surface 50 of the proximal head 24 may be roughened or provided with surface texturing to facilitate locking engagement of the proximal head 24 to the anchor hole 12.

The exemplary bone anchor 24 may include a distal portion 52 that is configured to engage bone. For example, the distal portion 52 of the bone anchor 14 may be threaded or include other structures or features configured to anchor the distal portion in bone.

The number of annular bores 30 provided within an anchor hole 12 may be varied depending on, for example, the size of the plate and the type of anchor employed. In addition, the structure of the annular bores 30, e.g., the size, shape and orientation of the stepped surfaces 32 and annular peaks 34, may also be varied. In the illustrated embodiment, each bore is commonly configured, e.g., commonly sized, shaped and oriented. One skilled in the art will appreciate an anchor hole may include one or more distinctly configured annular bores. Moreover, a bone fixation plate may be provided with differently configured anchor holes 12 and may include one or more anchor holes employing conventional locking mechanisms, such as, for example, a threaded connection or friction fit.

In other exemplary embodiments, one or more of the locking mechanism disclosed herein may be provided on the bone anchor. For example, the locking mechanism may comprise a plurality of concentric annular bores 30 formed on the outer surface 84 of the proximal head 82 of an exemplary bone anchor 80, as illustrated in FIG. 9. At least one of the plurality of concentric annular bores 30 may be sized and shaped to engage the inner surface 92 of an anchor hole 90, in a manner analogous to the anchor hole embodiments described above. For example, the plurality of concentric annular bores 30 may provide the outer surface 84 of the proximal head 82 of the bone anchor 80 with a generally stepped-shaped configuration. In particular, one or more of the bores 30 may include a radially extending step surface 32 that terminates at a radially outer, annular-shaped peak 34. Each peak 34 can provide an engagement surface for grippingly engaging the inner surface 92 of the anchor hole 90. To form the stepped shaped configuration, each peak 32 may have a diameter that is less than the diameter of the peak that is proximally adjacent to the peak. In such embodiments, the inner surface 92 of the anchor hole 90 may be generally smooth, although, other surface configurations, including a roughened surface, are contemplated.

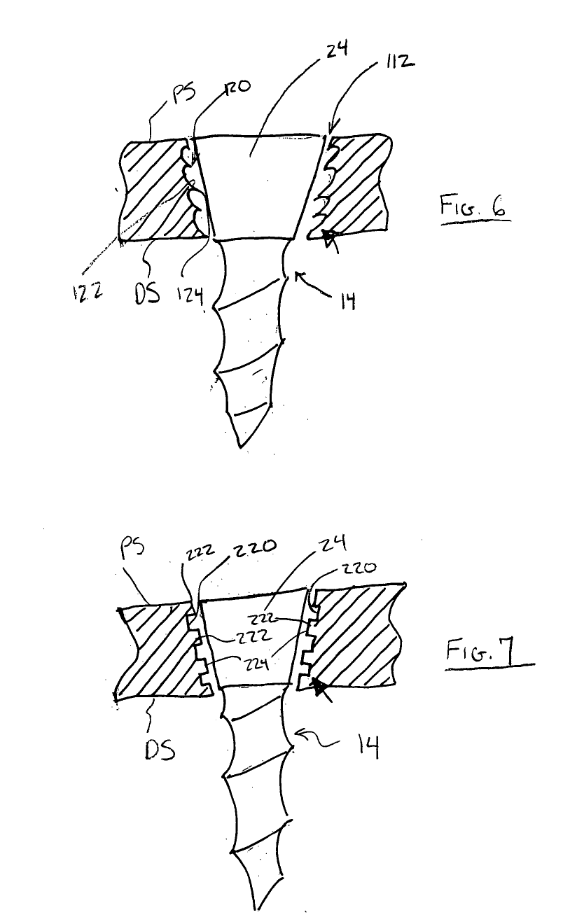

In alternative exemplary embodiments, a bushing, such as a polyaixal bushing, may be employed to securely affix the proximal head of the bone anchor to the bone fixation plate. FIG. 5 illustrates an exemplary embodiment of an annular polyaxial bushing 100 having a locking mechanism analogous to the locking mechanism described above in connection with anchor hole 12. In particular, the bushing 100 has an inner surface 20 that defines an anchor passage 22 for receiving a bone anchor, such as bone screw 14. A plurality of concentric annular bores 30 may provide the radial inner surface 20 of the exemplary bushing 100 with a generally stepped-shaped configuration to facilitate gripping engagement between the inner surface 20 and the proximal head 24 of the bone anchor 14.

The illustrated exemplary polyaxial bushing 100 is generally annular in cross-section and may include one or more slots or cutouts that allow for radial expansion of the bushing 100. The bushing 100 may have a generally spherically shaped radial outer surface 102. The radial outer surface 102 may be roughened by, for example, a plurality of circumferential ridges, or other surface texturing, that are configured to grippingly engage the smooth or roughened interior wall surface of an anchor hole. Radial expansion of bushing 100 expands the slot(s) in the bushing and presses the radial outer surface against the inner wall of the anchor hole for locking engagement between bushing 100 and bone fixation plate 10.

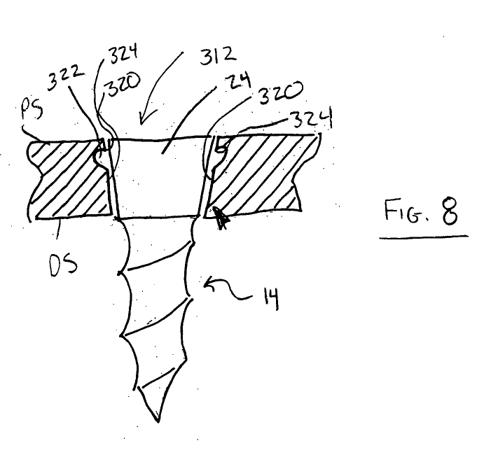

FIG. 6 illustrates an alternative embodiment of an anchor hole 112 including an inner surface 120 having a locking mechanism comprising one or more annular barbs 122 formed on the inner surface 120. The annular barbs 122 provide a step shaped geometry to inner surface 120 analogous to the step shaped geometry of the exemplary anchor hole 12 described above. The annular barbs 122 terminate radially at a point 124 that is oriented distally, i.e. in the direction of bone anchor insertion, to inhibit back-out of the bone anchor 14 from the plate 10. One skilled in the art will appreciate that one or more of the peaks 34 of the annular bores 30 described above may have analogous barbed shaped configuration.

FIG. 7 illustrates an alternative embodiment of an anchor hole 212 including an inner surface 220 having a locking mechanism comprising a plurality of annular ridges 222 formed on the inner surface 220. In the illustrated embodiment, each of the ridges 222 has a generally rectilinear cross section, although other cross sectional shapes are possible, including a radially inner engagement surface 224 for grippingly engaging the proximal head 24 of the bone anchor 14. The inner surface 220 may be tapered, as illustrated, such that the radially inner engagement surfaces 224 of the annular ridges 222 define a generally frusta-conical anchor passage 230 for receiving and engaging the bone anchor 14.

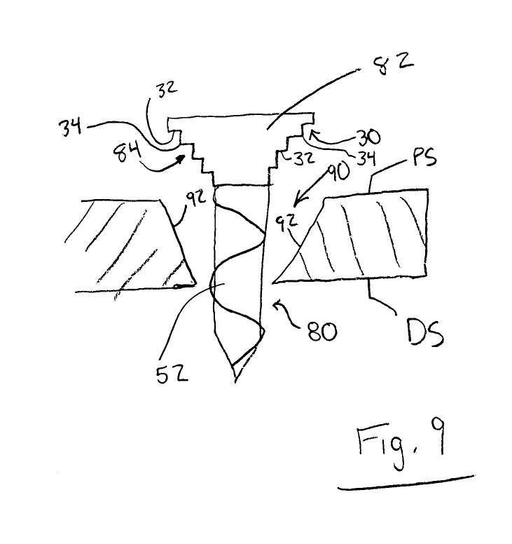

FIG. 8 illustrates a further alternative anchor hole 312 having a generally smooth, tapered inner surface 320 that includes a locking mechanism comprising an annular cut-out 322 configured to inhibit back-out of the bone anchor 14. The cut-out 322 has an arcuate cross section that terminates at a proximal end in a barbed-shaped edge 324 that can grippingly engage the outer surface of the proximal head 24 of the bone anchor 14.

While the bone fixation systems of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A bone fixation system comprising:
a bone anchor having a proximal head and a distal portion configured to engage bone, and
a plate having at least one hole for receiving the bone anchor, the at least one hole including a plurality of concentric annular bores formed in the plate, at least one of the plurality of concentric annular bores being sized and shaped to engage the proximal head of the bone anchor to facilitate coupling of the bone anchor to the plate.

2. The bone fixation system of claim 1, wherein the plurality of concentric annular bores includes a first bore having a first diameter and a second bore having a second diameter different from the first diameter.

3. The bone fixation system of claim 2, wherein the first bore is proximate the proximal surface of the plate relative to the second bore and the second diameter is less than the first diameter.

4. The bone fixation system of claim 1, where the proximal head is threadless.

5. The bone fixation system of claim 1, wherein the proximal head tapers toward the distal portion.

6. The bone fixation system of claim 1, wherein at least one of the plurality of concentric annular bores includes a barb to inhibit back-out of the bone anchor from the plate.

7. A bone plate comprising:
a body portion having at least one hole for receiving a bone anchor, the at least one hole including a plurality of annular concentric bores formed in the plate, at least one of the plurality of annular concentric bores being sized and shaped to engage the proximal head of the bone anchor to facilitate coupling of the bone anchor to the plate.

8. The bone plate of claim 7, wherein the plurality of concentric annular bores forms a generally frusta-conically shaped, stepped inner wall surface of the hole.

9. The bone plate of claim 8, wherein at least one bore of the plurality of concentric annular bores includes a barb to inhibit back-out of the bone anchor from the plate.

10. A bone plate comprising:
a body portion having at least one hole for receiving a bone anchor, the at least one hole having a generally stepped-shaped inner wall surface provided by a plurality of steps formed in the inner wall of the hole, each step having a annular peak, a plurality of the annular peaks being aligned in a generally frusta-conical shape.

11. The bone plate of claim 10, wherein the steps are symmetric about an axis of the hole.

12. A bone fixation system comprising:
a bone plate having a plurality of plate holes for receiving a bone anchor therein, and
a plurality of bone anchors for coupling the bone plate to bone, at least one of the bone anchors having a tapered proximal head and a distal portion configured to engage bone, the proximal head of the at least one anchor tapering toward the distal portion of the bone anchor,
at least one of the plates holes having a generally stepped-shaped inner wall surface provided by a plurality of steps formed in the inner wall of the at least one plate hole, a plurality of the steps having a generally annular peak, a plurality of the peaks being aligned in a generally frusta-conical shape to facilitate gripping engagement of the tapered proximal head of a bone anchor upon advancement of the bone anchor into the plate hole.

13. The bone fixation system of claim 12, wherein each annular peak of a plate hole defines a peak plane that intersects the axis of the hole.

14. The bone fixation system of claim 13, wherein at least one of the peak planes is oriented perpendicular to the axis of the hole.

15. The bone fixation system of claim 13, wherein at least one of the peak planes is oriented at angle other than perpendicular to the axis of the hole.

16. The bone fixation system of claim 13, wherein a plurality of the peak planes are parallel to one another.
